(19) United States
(12) Patent Application Publication
(10) Pub. No.: US 2015/0336885 A1
(43) Pub. Date: Nov. 26, 2015

(54) PROCESS FOR PREPARING 2,2'-SELENOBIARYL ETHERS OR 4,4'-SELENOBIARYL ETHERS USING SELENIUM DIOXIDE

(71) Applicants: Katrin Marie Dyballa, Recklinghausen (DE); Robert Franke, Marl (DE); Dirk Fridag, Haltern am See (DE); Siegfried R. Waldvogel, Gau-Algesheim (DE); Thomas Quell, Mainz (DE); Michael Mirion, Mainz (DE)

(72) Inventors: Katrin Marie Dyballa, Recklinghausen (DE); Robert Franke, Marl (DE); Dirk Fridag, Haltern am See (DE); Siegfried R. Waldvogel, Gau-Algesheim (DE); Thomas Quell, Mainz (DE); Michael Mirion, Mainz (DE)

(73) Assignee: Evonik Degussa GmbH, Essen (DE)

(21) Appl. No.: 14/720,063

(22) Filed: May 22, 2015

(30) Foreign Application Priority Data
May 26, 2014 (DE) 102014209974.9

(51) Int. Cl. C07C 391/02

(52) U.S. Cl. CPC C07C 391/02

(57) ABSTRACT

A process for preparing a 2,2′-selenobiaryl ether or a 4,4′-selenobiaryl ether, proceeds by a) adding a first phenol to the reaction mixture, b) adding a second phenol to the reaction mixture, c) adding selenium dioxide to the reaction mixture, d) adding a base having a pKb in the range from 8 to 11 to the reaction mixture, and e) adjusting the reaction temperature of the reaction mixture such that the first phenol and the second phenol are converted to said 2,2′-selenobiaryl ether or said 4,4′-selenobiaryl ether.

### FIELD OF THE INVENTION

The invention relates to a process for preparing 2,2′-selenobiaryl ethers or 4,4′-selenobiaryl ethers using selenium dioxide, and to a novel 2,2′-selenobiaryl ether or 4,4′-selenobiaryl ether.

### DISCUSSION OF THE BACKGROUND

2,2′-Selenobiaryl ethers or 4,4′-selenobiaryl ethers are a highly interesting and promising class of compounds. These compounds are currently being incorporated into particular complexes, particularly those containing manganese, but have great potential for further uses.

The term “phenols” is used as a generic term in this application and therefore also encompasses substituted phenols.

T. K. Paine describes a synthesis of 2,2′-selenobis(4,6-di-tert-butylphenol) using selenium dioxide. The preparation of 2,2′-selenobis(4,6-di-tert-butylphenol) is effected here in an acidic medium with addition of concentrated hydrochloric acid. The product is obtained with a yield of 25% (T. K. Paine et al., “Manganese complexes of mixed O, X, O-donor ligands (X=S or Se): synthesis, characterization and catalytic reactivity”, Dalton Trans., 2003, 15, 3136-3144).

It is particularly disadvantageous here that the yields are very low and therefore in need of improvement.

H. M. Lin describes a synthesis route for selenobiaryl ethers, which is effected over several stages. First of all, bromine has to be added onto the appropriate phenol, in order then to react the product with magnesium to give a Grignard reagent. The Grignard reagent can then react with the added selenium before the actual coupling to give the biaryl ether:

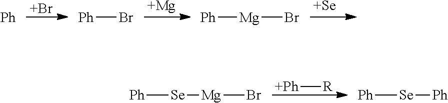

(H. M. Lin et al., “A novel and efficient synthesis of selenides”, ARKIVOC, 2012, viii, 146-156)

The product was obtained in a good yield, but this synthesis route is very complex, which makes it unattractive for industrial scale use. In this case, a multitude of synthesis steps are needed, the procedure for which is not uncritical in some cases, especially considering scale-up and using standards which are customary in industry. Moreover, this synthesis route gives rise to large amounts of waste products and solvents which have to be disposed of in a costly and inconvenient manner, one reason for which is the use of bromine.

### SUMMARY OF THE INVENTION

It was an object of the invention to provide a process which does not have the disadvantages described in connection with the related art. More particularly, a process by which 2,2′-selenobiaryl ethers or 4,4′-selenobiaryl ethers can be prepared selectively is to be provided, i.e. one in which the preparation gives rise to a minimum amount of by-products.

The process should also be usable on the industrial scale, and therefore have a minimum number of individual steps and intermediates.

This and other objects are achieved by the present invention which relates in one embodiment to a process for preparing a 2,2′-selenobiaryl ether or a 4,4′-selenobiaryl ether, comprising:

a) adding a first phenol to the reaction mixture,

b) adding a second phenol to the reaction mixture,

c) adding selenium dioxide to the reaction mixture,

d) adding a base having a pKb in the range from 8 to 11 to the reaction mixture, and

e) adjusting the reaction temperature of the reaction mixture such that the first phenol and the second phenol are converted to said 2,2′-selenobiaryl ether or said 4,4′-selenobiaryl ether.

### DETAILED DESCRIPTION OF THE INVENTION

Any ranges described below include all values and subvalues between the lower and upper limits of the range.

The present invention provides a process for preparing 2,2′-selenobiaryl ethers or 4,4′-selenobiaryl ethers, comprising the process steps of:

a) adding a first phenol to the reaction mixture,

b) adding a second phenol to the reaction mixture,

c) adding selenium dioxide to the reaction mixture,

d) adding a base having a pKb in the range from 8 to 11 to the reaction mixture,

e) adjusting the reaction temperature of the reaction mixture such that the first phenol and the second phenol are converted to a 2,2′-selenobiaryl ether or 4,4′-selenobiaryl ether.

Steps a) to d) can be conducted here in any sequence.

The process is not restricted to the components described above. Further constituents, for example solvents, may likewise be present in the reaction mixture.

If the base has more than one pKb, the pKb₁ should be considered. In the case of the invention, this has to be within the range from 8 to 11. The definition of pKa and pKb is sufficiently well known to those skilled in the art and can be found in the appropriate technical literature.

A problem with the use of selenium dioxide is that 2,2′-biphenols and the corresponding Pummerer ketone can be obtained as by-products in large amounts. In the case of an unfavorable reaction regime, it may even be the case that 2,2′-biphenols are the main product of the reaction. According to the objective of the invention, the aim, however, is to conduct the reaction specifically in such a way that the level of such by-products is reduced to a minimum.

Through addition of selenium dioxide as oxidizing agent, depending on the reaction conditions, 2,2′-biphenols or 2,2′-selenobiaryl ethers can be obtained as main products of the reaction (cf. Scheme 1).

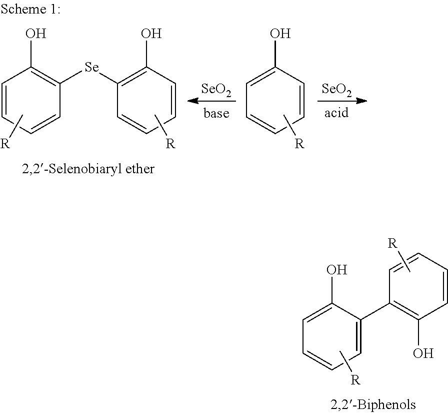

It has been found that the reaction can be shifted in the direction of the 2,2′-selenobiaryl ether in a controlled manner through addition of a base having a pKb in the range from 8 to 11.

Further advantages over the processes described in the related art are that it is not necessary to work with exclusion of moisture or oxygen. This constitutes a distinct advantage over other synthesis routes. This process stands out advantageously from the existing multistage synthesis routes.

Via the pKb values, the reaction can be steered in the direction of 2,2′-selenobiaryl ethers. As a result of predominant formation of the desired main product and reduction in the formation of higher molecular weight overoxidation products, the workup is distinctly simplified.

Unconverted reactants and solvents used can be recovered by distillation and used for further reactions. Thus, the process according to the invention fulfils the requirements for an economic industrial scale process.

Moreover, selenium dioxide is used in the process according to the invention. Selenium dioxide is a waste product from metal purification and ore refining. Thus, in the process claimed here, a waste product from other processes is reused with addition of value. This is an important topic especially against the background of the sustainability of processes.

In one variant of the process, the first phenol in process step a) is a compound of the general formula I:

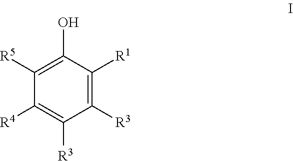

where R¹, R², R³, R⁴, R⁵ are each independently selected from:

—H, —(C₁-C₁₂)-alkyl, —O—(C₁-C₁₂)-alkyl, —(C₆-C₂₀)-aryl, —O—(C₆-C₂₀)-aryl, -halogen (such as Cl, F, Br, I), —OC═O—(C₁-C₁₂)-alkyl,

two adjacent radicals may additionally be joined to one another to form a condensed system,

where the alkyl and aryl groups mentioned may be substituted,

and at least R¹ or R⁵ is —H.

This process is used to prepare a 2,2′-selenobiaryl ether.

(C₁-C₁₂)-Alkyl and O—(C₁-C₁₂)-alkyl may each be unsubstituted or substituted by one or more identical or different radicals selected from:

(C₃C₁₂)-cycloalkyl, (C₃C₁₂)-heterocycloalkyl, (C₆-C₂₀)-aryl, fluorine, chlorine, cyano, formyl, acyl or alkoxycarbonyl.

(C₆-C₂₀)-Aryl and O—(C₆-C₂₀)-aryl may each be unsubstituted or substituted by one or more identical or different radicals selected from:

—H, —(C₁-C₁₂)-alkyl, —O—(C₁-C₁₂)-alkyl, —O—(C₆-C₂₀)-aryl, —(C₆-C₂₀)-aryl, -halogen (such as Cl, F, Br, I), —COO—(C₁-C₁₂)-alkyl, —CONH—(C₁-C₁₂)-alkyl, —(C₆-C₂₀)-aryl-CON[(C₁-C₁₂)-alkyl]₂, —CO—(C₁-C₁₂)-alkyl, —CO—(C₆-C₂₀)-aryl, —COOH, —OH, —SO₃H, —SO₃Na, —NO₂, —CN, —NH₂, —N[(C₁-C₁₂)-alkyl]₂.

In the context of the invention, the expression (C₁-C₁₂)-alkyl encompasses straight-chain and branched alkyl groups. Preferably, these groups are unsubstituted straight-chain or branched (C₁-C₈)-alkyl groups and most preferably (C₁-C₆)-alkyl groups. Examples of (C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The elucidations relating to the expression —(C₁-C₁₂)-alkyl also apply to the alkyl groups in —O—(C₁-C₁₂)-alkyl, i.e. in —(C₁-C₁₂)-alkoxy. Preferably, these groups are unsubstituted straight-chain or branched —(C₁-C₆)-alkoxy groups.

Substituted (C₁-C₁₂)-alkyl groups and substituted (C₁-C₁₂)-alkoxy groups may have one or more substituents, depending on their chain length. The substituents are preferably each independently selected from:

—(C₃-C₁₂)-cycloalkyl, —(C₃-C₁₂)-heterocycloalkyl, —(C₆-C₂₀)-aryl, fluorine, chlorine, cyano, formyl, acyl or alkoxycarbonyl.

In one variant of the process, R¹, R², R³, R⁴, R⁵ are each independently selected from:

—H, —(C₁-C₁₂)-alkyl, —O—(C₁-C₁₂)-alkyl, —(C₆-C₂₀)-aryl, —O—(C₆-C₂₀)-aryl,

where the alkyl and aryl groups mentioned may be substituted,

and at least R¹ or R⁵ is —H.

In one variant of the process, R¹, R², R³, R⁴, R⁵ are each independently selected from:

—H, —(C₁-C₁₂)-alkyl,

where the alkyl and aryl groups mentioned may be substituted,

and at least R¹ or R⁵ is —H.

In one variant of the process, R¹, R³, R⁵ are each independently selected from:

—H, —(C₁-C₁₂)-alkyl,

where the alkyl groups mentioned may be substituted,

and at least R¹ or R⁵ is —H.

In one variant of the process, R² and R⁴ are each —H.

In one variant of the process, the second phenol in process step b) is a compound of the general formula II:

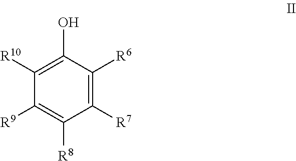

where R⁶, R⁷, R⁸, R⁹, R¹⁰ are each independently selected from:

—H, —(C₁-C₁₂)-alkyl, —O—(C₁-C₁₂)-alkyl, —(C₆-C₂₀)-aryl, —O—(C₆-C₂₀)-aryl, -halogen (such as Cl, F, Br, I), —OC═O—(C₁-C₁₂)-alkyl,

two adjacent radicals may additionally be joined to one another to form a condensed system,

where the alkyl and aryl groups mentioned may be substituted,

and at least R⁶ or R¹⁰ is —H.

In one variant of the process, R⁶, R⁷, R⁸, R⁹, R¹⁰ are each independently selected from:

—H, —(C₁-C₁₂)-alkyl, —O—(C₁-C₁₂)-alkyl, —(C₆-C₂₀)-aryl, —O—(C₆-C₂₀)-aryl,

where the alkyl and aryl groups mentioned may be substituted,

and at least R⁶ or R¹⁰ is —H.

In one variant of the process, R⁶, R⁷, R⁸, R⁹, R¹⁰ are each independently selected from:

—H, —(C₁-C₁₂)-alkyl,

where the alkyl and aryl groups mentioned may be substituted,

and at least R⁶ or R¹⁰ is —H.

In one variant of the process, R⁶, R⁸, R¹⁰ are each independently selected from:

—H, —(C₁-C₁₂)-alkyl,

where the alkyl groups mentioned may be substituted,

and at least R⁶ or R¹⁰ is —H.

In one variant of the process, R⁷ and R⁹ are each —H.

In one variant of the process, the first phenol corresponds to the second phenol.

This variant is thus a homo-coupling of two identical phenols which are joined via the selenium.

In one variant of the process, the selenium dioxide is added in process step c) in a molar ratio based on the sum total of the first and second phenols within a range from 0.25 to 1.5.

Preference is given here to the range from 0.25 to 0.9, and particular preference to the range from 0.4 to 0.7.

In one variant of the process, the base in process step d) is selected from:

pyridine, quinoline.

Preference is given here to pyridine.

In one variant of the process, the base in process step d) is used as solvent.

In one variant of the process, the reaction mixture is adjusted in process step e) to a temperature in the range from 0° C. to 100° C.

Preference is given here to the range from 20° C. to 90° C., and particular preference to the range from 30° C. to 80° C.

In one variant of the process, the temperature set in process step e) is maintained over a period in the range from 1 hour to 48 hours.

Preference is given here to the range from 1 hour to 24 hours, and particular preference to the range from 2 hours to 10 hours.

As well as the process, a novel 2,2′-selenobiaryl ether is also claimed.

Compound of the Formula:

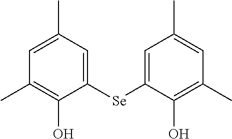

In one variant of the process, the first phenol in process step a) is a compound of the general formula III:

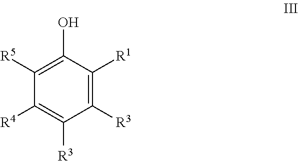

where R¹, R², R³, R⁴, R⁵ are each independently selected from:

—H, —(C₁-C₁₂)-alkyl, —O—(C₁-C₁₂)-alkyl, —(C₆-C₂₀)-aryl, —O—(C₆-C₂₀)-aryl, -halogen (such as Cl, F, Br, I), —OC═O—(C₁-C₁₂)-alkyl,

two adjacent radicals may additionally be joined to one another to form a fused system,

where the alkyl and aryl groups mentioned may be substituted,

and R³ is —H.

This process is used to prepare a 4,4′-selenobiaryl ether.

In one variant of the process, R¹, R², R³, R⁴, R⁵ are each independently selected from:

—H, —(C₁-C₁₂)-alkyl, —O—(C₁-C₁₂)-alkyl, —(C₆-C₂₀)-aryl, —O—(C₆-C₂₀)-aryl,

where the alkyl and aryl groups mentioned may be substituted,

and R³ is —H.

In one variant of the process, R¹, R², R³, R⁴, R⁵ are each independently selected from:

—H, —(C₁-C₁₂)-alkyl,

where the alkyl and aryl groups mentioned may be substituted,

and R³ is —H.

In one variant of the process, R¹, R³, R⁵ are each independently selected from:

—H, —(C₁-C₁₂)-alkyl,

where the alkyl groups mentioned may be substituted,

and R³ is —H.

In one variant of the process R² and R⁴ are each —H.

In one variant of the process, the second phenol in process step b) is a compound of the general formula IV:

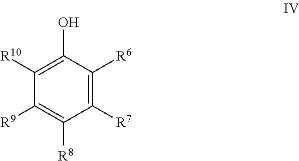

where R⁶, R⁷, R⁸, R⁹, R¹⁰ are each independently selected from:

—H, —(C₁-C₁₂)-alkyl, —O—(C₁-C₁₂)-alkyl, —(C₆-C₂₀)-aryl, —O—(C₆-C₂₀)-aryl, -halogen (such as Cl, F, Br, I), —OC═O—(C₁-C₁₂)-alkyl,

two adjacent radicals may additionally be joined to one another to form a fused system,

where the alkyl and aryl groups mentioned may be substituted,

and R⁸ is —H.

In one variant of the process, R⁶, R⁷, R⁸, R⁹, R¹⁰ are each independently selected from:

—H, —(C₁-C₁₂)-alkyl, —O—(C₁-C₁₂)-alkyl, —(C₆-C₂₀)-aryl, —O—(C₆-C₂₀)-aryl,

where the alkyl and aryl groups mentioned may be substituted,

and R⁸ is —H.

In one variant of the process, R⁶, R⁷, R⁸, R⁹, R¹⁰ are each independently selected from:

—H, —(C₁-C₁₂)-alkyl,

where the alkyl and aryl groups mentioned may be substituted,

and R⁸ is —H.

In one variant of the process, R⁶, R⁸, R¹⁰ are each independently selected from:

—H, —(C₁-C₁₂)-alkyl,

where the alkyl groups mentioned may be substituted,

and R⁸ is —H.

In one variant of the process, R⁷ and R⁹ are each —H.

In one variant of the process, the first phenol corresponds to the second phenol.

This variant is thus a homo-coupling of two identical phenols which are joined via the selenium.

In one variant of the process, the selenium dioxide, in process step c), is added in a molar ratio, based on the sum total of the first and second phenols, within a range from 0.25 to 1.5.

Preference is given here to the range from 0.25 to 0.9, and particular preference to the range from 0.4 to 0.7.

In one variant of the process, the base in process step d) is selected from:

pyridine, quinoline.

Preference is given here to pyridine.

In one variant of the process, the base is used as solvent in process step d).

In one variant of the process, the reaction mixture is set in process step e) to a temperature in the range from 0° C. to 100° C.

Preference is given here to the range from 20° C. to 90° C., and particular preference to the range from 30° C. to 80° C.

In one variant of the process, the temperature set in process step e) is maintained over a period in the range from 1 hour to 48 hours.

Preference is given here to the range from 1 hour to 24 hours, and particular preference to the range from 2 hours to 10 hours.

As well as the process, a novel 4,4′-selenobiaryl ether is also claimed.

Compound of the Formula:

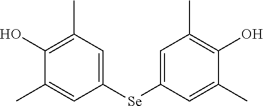 [SMILES: Cc1cc([Se]c2cc(C)c(O)c(C)c2)cc(C)c1O]

Having generally described this invention, a further understanding can be obtained by reference to certain specific examples which are provided herein for purposes of illustration only, and are not intended to be limiting unless otherwise specified.

### Examples

### Analysis

### NMR Spectroscopy

The mass spectroscopy studies were conducted on multi-nucleus resonance spectrometers of the AC 300 or AV II 400 type from Bruker, Analytische Messtechnik, Karlsruhe. The solvent used was CDCl₃. The ¹H and ¹³C spectra were calibrated according to the residual content of undeuterated solvent using the NMR Solvent Data Chart from Cambridge Isotopes Laboratories, USA. Some of the ¹H and ¹³C signals were assigned with the aid of H,H-COSY, H,H-NOESY, H,C-HSQC and H,C-HMBC spectra. The chemical shifts are reported as δ values in ppm. For the multiplicities of the NMR signals, the following abbreviations were used: s (singlet), bs (broad singlet), d (doublet), t (triplet), q (quartet), m (multiplet), dd (doublet of doublets), dt (doublet of triplets), tq (triplet of quartets). All coupling constants J were reported in hertz (Hz) together with the number of bonds covered. The numbering given in the assignment of signals corresponds to the numbering shown in the formula schemes, which does not necessarily have to correspond to IUPAC nomenclature.

### General Procedure

8.2 mmol of the particular phenol are dissolved in the appropriate solvent (8.2 M). The reaction mixture is heated, and 4.9 mmol of selenium dioxide are added while stirring. The solvent is distilled under reduced pressure (temperature<70° C.). A frit is prepared with 2.5 cm of silica gel (at the bottom) and 2.5 cm of zeolite (at the top). The distillation residue is taken up in the eluent and applied to the filtration column. Cyclohexane:ethyl acetate (95:5) is used to wash the product off the frit and collect it in fractions. The fractions containing product are combined and freed of the eluent by distillation.

The fractions obtained are recrystallized from 95:5 cyclohexane:ethyl acetate. For this purpose, the solid residue is dissolved at 50° C., and insoluble residues are filtered off using a glass frit. The reaction product crystallizes out of the saturated solution at room temperature overnight. The resulting crystals are washed once again with cold cyclohexane.

The structural formula shows the main product obtained in each reaction.

### 3,3′,5,5′-Tetramethylbiphenyl-2,2′-diol

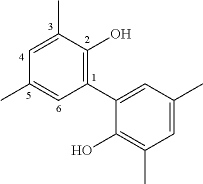

The reaction is conducted according to the general procedure in a screw-top test tube. For this purpose, 1.00 g (8.2 mmol, 1.0 equiv.) of 2,4-dimethylphenol and 0.54 g (4.9 mmol, 0.6 equiv.) of selenium dioxide are dissolved and heated in 1 ml of acid. The product is obtained as a beige crystalline solid.

¹H NMR (300 MHz, CDCl₃):

δ (ppm)=7.00 (s, 2H, 6-H), 6.87 (s, 2H, 4-H), 5.07 (s, 2H, OH), 2.27 (s, 12H, 3-CH₃, 5-CH₃).

¹³C NMR (75 MHz, CDCl₃):

δ (ppm)=149.2 (C-2), 132.1 (C-4), 130.0 (C-5), 128.5 (C-6), 125.1 (C-3), 122.1 (C-1), 20.4 (5-CH₃), 16.2 (3-CH₃).

### Bis(3,5-dimethyl-2-hydroxyphenyl)selenium

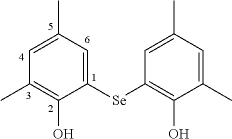

The reaction is conducted according to the general procedure in a screw-top test tube. For this purpose, 1.00 g (8.2 mmol, 1.0 equiv.) of 2,4-dimethylphenol and 0.54 g (4.9 mmol, 0.6 equiv.) of selenium dioxide are dissolved and heated in 1 ml of pyridine. The product is obtained as a colourless crystalline solid.

In a 250 ml round-bottom flask, 49.9 g of selenium dioxide (413 mmol) in 100 ml of pyridine were heated to 55° C. with the aid of an oil bath. Subsequently, 25 ml of 2,4-dimethylphenol (206 mmol) were added thereto and the temperature was maintained for seven-and-a-half hours. After the reaction had ended, the mixture was diluted with 400 ml of ethyl acetate and filtered. The organic phase was washed with water and dried over magnesium sulphate. The pyridine was removed by distillation and the residue was dissolved again in ethyl acetate and washed with 10% hydrochloric acid and water, in order to remove residues of pyridine. The organic phase was dried with magnesium sulphate and freed of solvent under reduced pressure. The crude product thus obtained was heated under reflux in 400 ml of cyclohexane. After cooling to room temperature, the product crystallized. After one day, the product was filtered off, and the filtrate was concentrated by half the volume, in order to be crystallized again at 4° C.

Yield: 18.559 g (57.8 mmol), 56%

¹H NMR (400 MHz, CDCl₃):

δ (ppm)=7.12 (s, 2H, 6-H), 6.91 (s, 2H, 4-H), 5.97 (s, 2H, OH), 2.23 (s, 6H, 3-CH₃) 2.23 (s, 6H, 5-CH₃).

¹³C NMR (100 MHz, CDCl₃):

δ (ppm)=151.7 (C-2), 133.2 (C-3), 133.1 (C-5), 130.4 (C-4), 124.2 (C-6), 114.9 (C-1), 20.3 (5-CH₃), 16.5 (3-CH₃).

⁷⁷Se NMR (76 MHz, CDCl₃):

δ (ppm)=163.36 ppm.

### Bis(3-tert-butyl-5-methyl-2-hydroxyphenyl)selenium

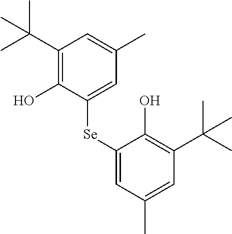 [SMILES: Cc1cc([Se]c2cc(C)cc(C(C)(C)C)c2O)c(O)c(C(C)(C)C)c1]

The reaction is conducted according to the general procedure in a screw-top test tube. For that purpose, 1.32 g (8.0 mmol, 1.0 equiv.) of 2-tert-butyl-4-methylphenol and 0.54 g (4.9 mmol, 0.6 equiv.) of selenium dioxide were dissolved and heated in 1 ml of pyridine.

¹H NMR (300 MHz, CDCl₃):

δ (ppm)=7.15 (s, 2H, 6-H), 7.05 (s, 2H, 4-H), 5.07 (s, 2H, OH), 2.21 (s, 6H, 5-CH₃), 2.21 (s, 18H, 3-C(CH₃)_{3.}

¹³C NMR (75 MHz, CDCl₃):

δ (ppm)=152.1, 136.4, 133.4, 120.1, 129.5, 117.2, 35.1, 29.6, 20.8.

### 3,3′-Di-tert-butyl-5,5′-dimethylbiphenyl-2,2′-diol

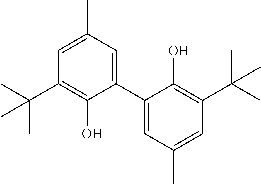 [SMILES: Cc1cc(-c2cc(C)cc(C(C)(C)C)c2O)c(O)c(C(C)(C)C)c1]

The reaction is conducted according to the general procedure in a screw-top test tube. For that purpose, 5.00 g (30.5 mmol, 1.0 equiv.) of 2-tert-butyl-4-methylphenol and 2.03 g (18.3 mmol, 0.6 equiv.) of selenium dioxide were dissolved and heated in 5 ml of acetic acid.

¹H NMR (400 MHz, CDCl₃):

δ (ppm)=7.17 (d, J=2.2 Hz, 2H), 6.91 (d, J=2.2 Hz, 2H), 5.19 (s, 2H), 2.33 (s, 6H), 1.45 (s, 18H).

¹³C NMR (75 MHz, CDCl₃):

δ (ppm)=149.9, 137.0, 129.7, 128.9, 128.6, 122.7, 35.0, 29.8, 27.0.

### Bis(3,5-Di-tert-butyl-2-hydroxyphenyl)selenium

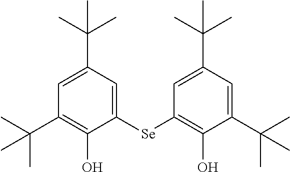 [SMILES: CC(C)(C)c1cc([Se]c2cc(C(C)(C)C)cc(C(C)(C)C)c2O)c(O)c(C(C)(C)C)c1]

The reaction is conducted according to the general procedure in a screw-top test tube. For that purpose, 1.67 g (8.2 mmol, 1.0 equiv.) of 2,4-di-tert-butylphenol and 0.55 g (4.9 mmol, 0.6 equiv.) of selenium dioxide were dissolved and heated in 1 ml of pyridine.

¹H NMR (400 MHz, CDCl₃):

δ (ppm)=7.31 (d, J=2.4 Hz, 2H), 7.29 (d, J=2.4), 6.29 (s, 2H), 1.42 (s, 18H), 1.24 (s, 18H).

¹³C NMR (75 MHz, CDCl₃):

δ (ppm)=151.7, 143.5, 135.8, 129.8, 125.6, 117.2, 35.4, 34.4, 31.6, 29.7.

### bis(3-Chloro-6-hydroxy-5-isopropyl-2-methylphenyl)selenium

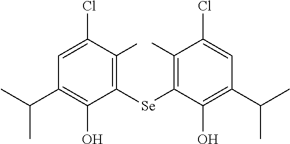 [SMILES: Cc1c(Cl)cc(C(C)C)c(O)c1[Se]c1c(C)c(Cl)cc(C(C)C)c1O]

In a 10 ml round-bottom flask, 1.0 g of chlorothymol (54 mmol) were dissolved in 7.5 ml of pyridine, 0.601 g of selenium dioxide (54 mmol) were added and the mixture was heated to 55° C. in an oil bath. After seven days, the reaction solution was diluted with 50 ml of ethyl acetate and filtered. The organic phase was first washed twice with 40 ml each time of 10% hydrochloric acid and twice with 40 ml each time of water, and dried over magnesium sulphate. The crude product obtained after distillation of the solvent was purified by means of column chromatography: the length of the column was 10 cm with a diameter of 4 cm. The eluent used was cyclohexane/ethyl acetate in a ratio of 95/5. The crude product thus obtained was heated under reflux in 20 ml of cyclohexane. Good crystallization was achievable only by very gentle cooling in an oil bath. The product was filtered off and washed with cold cyclohexane. The filtrate was concentrated and crystallized again at 4° C. within two days. After the solids had been filtered off, the filtrate was concentrated again and crystallized at 4° C. for seven days.

In order to obtain suitable single crystals for x-ray structure analysis, 200 mg of the product were dissolved in 0.5 ml of dichloromethane and blanketed with 10 ml of cyclohexane. After only one day, crystal growth was observed in the region of the former phase boundary. After seven days, it was possible to remove suitable single crystals.

Yield: 422 mg (0.9 mmol), 35%

¹H NMR: (400 MHz, CDCl₃) δ [ppm]=1.20 (d, J=6.9 Hz, 12H), 2.42 (s, 6H), 3.21 (hept, J=7 Hz, 2H), 6.37 (s, 2H), 7.18 (s, 2H)

¹³C NMR: (100 MHz, CDCl₃) δ [ppm]=20.76, 22.41, 27.96, 117.49, 126.39, 128.45, 134.16, 136.80, 152.70

Melting range: 175.3-175.8° C.

### bis(3-Chloro-6-hydroxy-5-methylphenyl)selenium

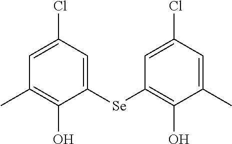

In a 10 ml round-bottom flask, 650 mg of 4-chloro-2-methylphenol (45 mmol) were dissolved in 6.3 ml of pyridine, 506 mg of selenium dioxide (45 mmol) were added and the mixture was heated to 55° C. in an oil bath. After 10 days, the reaction solution was diluted with 50 ml of ethyl acetate and filtered. The organic phase was first washed twice with 40 ml each time of 10% hydrochloric acid and twice with 40 ml each time of water. After drying over magnesium sulphate, the solvent was removed by distillation and the crude product was purified by means of column chromatography. This was done using an automated column system from BÜCHI-Labortechnik GmbH, Essen. The column length was 16 cm and the diameter 6 cm. The eluent used was cyclohexane/ethyl acetate, working with an ethyl acetate gradient: 1-5% (over 15 min), 3-20% (over 20 min), 20-60% (20 min). The pumping rate was 50 ml/min. The product obtained was dissolved in dichloromethane with a 5% addition of methanol and blanketed with cyclohexane, in order to enable crystallization at the interface. Colourless, acicular crystals were obtained.

Yield: 393 mg (1.0 mmol), 48%

¹H NMR: (400 MHz, CDCl₃) δ [ppm]=2.25 (s, 6H), 7.09-7.11 (m, 2H), 7.19-7.22 (m, 2H)

¹³C NMR: (100 MHz, CDCl₃) δ [ppm]=16.67, 125.57, 126.32, 126.91, 131.99, 132.24, 152.60

### bis(2-Hydroxy-3-methoxy-5-methylphenyl)selenium

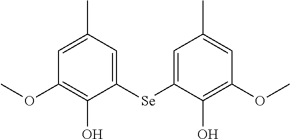

In a 10 ml round-bottom flask, 700 mg of 4-methylguaiacol (51 mmol) were dissolved in 7.1 ml of pyridine, 0.856 g of selenium dioxide (77 mmol) were added and the mixture was heated in an oil bath to 55° C. After four days, the reaction solution was diluted with 50 ml of ethyl acetate and filtered. The organic phase was first washed twice with 40 ml each time of 10% hydrochloric acid and twice with 40 ml each time of water. After drying over magnesium sulphate, the solvent was removed by distillation and the crude product was purified by means of column chromatography. This was done using an automated column system from BÜCHI-Labortechnik GmbH, Essen. The column length was 16 cm and the diameter 6 cm. The eluent used was cyclohexane/ethyl acetate, and an ethyl acetate gradient of 1-20% over 80 minutes was employed. The pumping rate was 50 ml/min.

In order to obtain suitable single crystals for x-ray structure analysis, 100 mg of the product were dissolved in 0.3 ml of dichloromethane and blanketed with 7 ml of cyclohexane. Platelets composed of clear, pale yellowish crystals were obtained.

Yield: 167 mg (0.4 mmol), 19%

¹H NMR: (400 MHz, CDCl₃) δ [ppm]=2.22 (s, 6H), 3.85 (s, 6H), 6.26 (s, 2H), 6.64 (d, J=1.6 Hz, 2H), 6.79 (dd, J1=1.8 Hz, J2=0.7 Hz, 2H)

¹³C NMR: (100 MHz, CDCl₃) δ [ppm]=21.12, 56.22, 112.87, 114.96, 126.89, 130.30, 143.35, 156.52

Melting range: 146.5-146.8° C.

### bis(3,5-Dimethyl-4-hydroxyphenyl)selenium

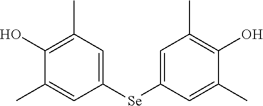 [SMILES: Cc1cc([Se]c2cc(C)c(O)c(C)c2)cc(C)c1O]

In a culture tube, 500 mg (4.1 mmol) of 2,6-dimethylphenol were dissolved in 5.7 ml of pyridine and admixed with 250 mg (2.2 mmol). The mixture was heated in a steel block at 55° C. After 24 hours, the reaction mixture was diluted with 40 ml of dichloromethane and filtered. The filtrate was washed twice with 30 ml each time of hydrochloric acid (10%) and 10 twice with 30 ml each time of water. The organic phase was removed, dried with magnesium sulphate and freed of the solvent. The crude product thus obtained was purified by means of column chromatography. This was done using an automated column system from BÜCHI-Labortechnik GmbH, Essen. The column length was 16 cm and the diameter 6 cm. The eluent used was cyclohexane/ethyl acetate, and an ethyl acetate gradient was employed: 5-10% (over 20 min), 25-100% (over 5 min). The product thus obtained was dissolved in 20 ml of cyclohexane at boiling. After cooling to room temperature, yellowish needles formed.

Yield: 342 mg (1.1 mmol), 52%

¹H NMR: (300 MHz, CDCl₃) δ [ppm]=2.21 (s, 12H), 4.62 (s, 2H), 7.15 (s, 4H)

¹³C NMR: (75 MHz, CDCl₃) δ [ppm]=15.92, 121.48, 124.21, 133.71, 152.03

Melting range: 220.7-222.1° C.

### 3,3′,5,5′-Tetra-tert-butylbiphenyl-2,2′-diol

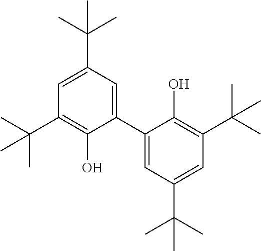 [SMILES: CC(C)(C)c1cc(-c2cc(C(C)(C)C)cc(C(C)(C)C)c2O)c(O)c(C(C)(C)C)c1]

The reaction is conducted according to the general procedure in a screw-top test tube. For that purpose, 307 mg (1.5 mmol, 1.0 equiv.) of 2,4-di-tert-butylphenol and 99 mg (0.8 mmol, 0.6 equiv.) of selenium dioxide were dissolved and heated in 0.5 ml of acetic acid.

¹H NMR (400 MHz, CDCl₃):

δ (ppm)=7.39 (d, J=2.4 Hz, 2H), 7.11 (d, J=2.4, 2H), 5.21 (s, 2H), 1.45 (s, 18H), 1.32 (s, 18H).

¹³C NMR (75 MHz, CDCl₃):

δ (ppm)=149.9, 143.0, 125.4, 124.9, 122.4, 35.4, 34.6, 31.7, 29.8.

The results of the above-described reaction, and variations thereof, are shown in the tables which follow. The processes according to the invention are identified here by *.

The following compound classes are specified in detail in the tables:

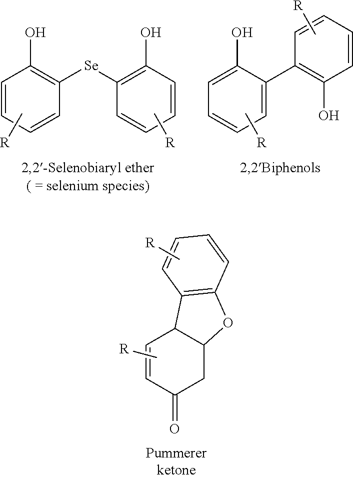

TABLE 1a
Oxidative coupling of 2,4-dimethylphenol
Basic conditions

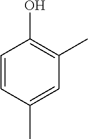

| | T | t | | Pummerer | Biphenol | Selenium |
|---|---|---|---|---|---|---|
| Solvent | [° C.] | [h] | pKb | ketone [%] | [%] | species [%] |
| Pyridine* | 60 | 5 | 8.9 | — | — | 79.1 |
| Pyridine* | 85 | 5 | 8.9 | 2.6 | 13.1 | 59.6 |
| Pyridine* | 100 | 0.5 | 8.9 | 1.9 | 11.0 | 39.9 |
| Quinoline* | 60 | 7 | 9.2 | 0.6 | 1.9 | 28.6 |
| Triethylamine | 80 | 4 | 3.3 | — | — | 1.8 |
| (dry) | | | | | | |
| DMF | 85 | 5 | −1.1 | 4.2 | 19.1 | 18.8 |

A further nitrogen base used was 4-dimethylaminopyridine (pKb=4.8). The reaction time studied was 1 h, and neither the biphenol nor the selenium species were detectable by gas chromatography.

It can be inferred from Table 1a that, under the conditions of the invention, the desired 2,2′-selenobiaryl ether is always obtained as the main product, in a distinct excess relative to the by-products, and in a good yield.

TABLE 1b
Oxidative coupling of 2,4-dimethylphenol
Acidic conditions

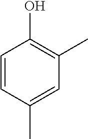

| | T | t | | Pummerer | Biphenol | Selenium |
|---|---|---|---|---|---|---|
| Solvent | [° C.] | [h] | pKa | ketone [%] | [%] | species [%] |
| Acetic acid | 85 | 5 | 4.8 | 4.5 | 74.8 | 1.98 |
| Acetic acid | 60 | 1.5 | 4.8 | 1.8 | 39.8 | 8.0 |
| Trifluoroacetic acid/ | 85 | 5 | 0.23/4.8 | 2.5 | 77.8 | 1.4 |
| acetic acid (3:1) | | | | | | |
| Formic acid | 60 | 2 | 3.8 | 1.8 | 85.4 | — |
| Methanesulphonic acid | 85 | 5 | −2.6 | — | 3.9 | 6.1 |
| p-Toluenesulphonic acid | 85 | 5 | −2.8 | — | 15.0 | — |

It can be inferred from Table 1b that the biphenol is obtained as the main product in each case under acidic conditions. The sole exception is methanesulphonic acid, although the yield of the selenium species is very low here.

TABLE 2a
Oxidative coupling of 2,4-di-tert-butylphenol
Basic conditions

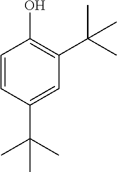

| | T | t | | Biphenol | Selenium |
|---|---|---|---|---|---|
| Solvent | [° C.] | [h] | pKb | [%] | species [%] |
| Pyridine* | 40 | 24 | 8.9 | 20.6 | 46.8 |
| Pyridine* | 60 | 7 | 8.9 | 10.1 | 30.4 |

Under the basic conditions of the invention, the selenium species again forms as the main product of the reaction.

TABLE 2b
Oxidative coupling of 2,4-di-tert-butylphenol
Acidic conditions

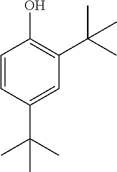

| | T | t | | Biphenol | Selenium |
|---|---|---|---|---|---|
| Solvent | [° C.] | [h] | pKa | [%] | species [%] |
| Acetic acid | 50 | 18 | 4.8 | 29.5 | 25.2 |
| Acetic acid | 85 | 1 | 4.8 | 25.9 | 23.1 |
| Acetic acid | 105 | 0.2 | 4.8 | 75.1 | 2.6 |
| Formic acid | 70 | 1 | 3.8 | 46.9 | 7.6 |

Under acidic conditions, in contrast, the unwanted biphenol is the main product of the reaction.

TABLE 3a
Oxidative coupling of 2-tert-butyl-4-methylphenol
Basic conditions

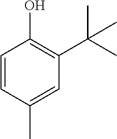

| | T | t | | Biphenol | Selenium |
|---|---|---|---|---|---|
| Solvent | [° C.] | [h] | pKa | [%] | species [%] |
| Pyridine* | 40 | 24 | 8.9 | 7.2 | 61.5 |
| Pyridine* | 60 | 7 | 8.9 | 1.6 | 32.2 |
| Pyridine* | 85 | 1.5 | 8.9 | 6.1 | 32.5 |
| Pyridine* | 100 | 0.5 | 8.9 | 4.5 | 28.9 |

From Table 3a too, it is again clear that the basic conditions of the invention lead to the desired selenium species. This is obtained in a distinct excess over the unwanted biphenol.

TABLE 3b
Oxidative coupling of 2-tert-butyl-4-methylphenol
Acidic conditions

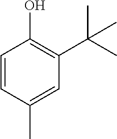

| | T | t | | Biphenol | Selenium |
|---|---|---|---|---|---|
| Solvent | [° C.] | [h] | pKa | [%] | species [%] |
| Acetic acid | 50 | 18 | 4.8 | 34.2 | 19.8 |
| Acetic acid | 85 | 1.5 | 4.8 | 63.7 | 4.0 |
| Acetic acid | 100 | 0.4 | 4.8 | 53.2 | 2.1 |

Under acidic conditions, in contrast, the desired selenium species is again only the by-product.

The results summarized in Tables 1a to 3b show clearly that the process according to the invention fulfils the objective defined above. The process according to the invention is a synthesis route by which 2,2′-selenobiaryl ethers can be prepared selectively, in a good yield. In addition, the process according to the invention can also be implemented on the industrial scale. The phenols are converted directly to the corresponding 2,2′-selenobiaryl ethers in a single reaction step.

German patent application 102014209974.9 filed May 26, 2014, is incorporated herein by reference.

Numerous modifications and variations on the present invention are possible in light of the above teachings. It is therefore to be understood that within the scope of the appended claims, the invention may be practiced otherwise than as specifically described herein.

1. A process for preparing a 2,2′-selenobiaryl ether or a 4,4′-selenobiaryl ether, comprising:
a) adding a first phenol to the reaction mixture,
b) adding a second phenol to the reaction mixture,
c) adding selenium dioxide to the reaction mixture,
d) adding a base having a pKb in the range from 8 to 11 to the reaction mixture, and
e) adjusting the reaction temperature of the reaction mixture such that the first phenol and the second phenol are converted to said 2,2′-selenobiaryl ether or said 4,4′-selenobiaryl ether.

2. The process according to claim 1, wherein the first phenol in process step a) is a compound of the general formula I: 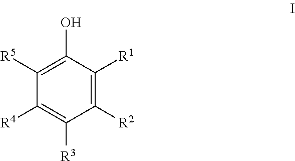
wherein R¹, R², R³, R⁴, R⁵ are each independently selected from the group consisting of:
—H, —(C₁-C₁₂)-alkyl, —O—(C₁-C₁₂)-alkyl, —(C₆-C₂₀)-aryl, —O—(C₆-C₂₀)-aryl, -halogen, and —OC═O—(C₁-C₁₂)-alkyl,
wherein two adjacent radicals are optionally joined to one another to form a condensed system,
wherein the alkyl and aryl groups mentioned are optionally substituted, and
wherein at least R¹ or R⁵ is —H.

3. The process according to claim 2, wherein R¹, R², R³, R⁴, R⁵ are each independently selected from the group consisting of:
—H, —(C₁-C₁₂)-alkyl, —O—(C₁-C₁₂)-alkyl, —(C₆-C₂₀)-aryl, and —O—(C₆-C₂₀)-aryl,
wherein the alkyl and aryl groups mentioned are optionally substituted, and
wherein at least R¹ or R⁵ is —H.

4. The process according to claim 2, wherein R¹, R³, R⁵ are each independently selected from the group consisting of:
—H, and —(C₁-C₁₂)-alkyl,
wherein the alkyl groups mentioned are optionally substituted, and
wherein at least R¹ or R⁵ is —H.

5. The process according to claim 1, wherein the second phenol in process step b) is a compound of the general formula II: 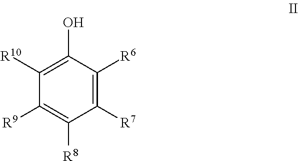
wherein R⁶, R⁷, R⁸, R⁹, R¹⁰ are each independently selected from the group consisting of:
—H, —(C₁-C₁₂)-alkyl, —O—(C₁-C₁₂)-alkyl, —(C₆-C₂₀)-aryl, —O—(C₆-C₂₀)-aryl, -halogen, and —OC═O—(C₁-C₁₂)-alkyl,
wherein two adjacent radicals are optionally joined to one another to form a condensed system,
wherein the alkyl and aryl groups mentioned are optionally substituted, and
wherein at least R⁶ or R¹⁰ is —H.

6. The process according to claim 5, wherein R⁶, R⁷, R⁸, R⁹, R¹⁰ are each independently selected from the group consisting of:
—H, —(C₁-C₁₂)-alkyl, —O—(C₁-C₁₂)-alkyl, —(C₆-C₂₀)-aryl, and —O—(C₆-C₂₀)-aryl,
wherein the alkyl and aryl groups mentioned are optionally substituted, and
wherein at least R⁶ or R¹⁰ is —H.

7. The process according to claim 5, wherein R⁶, R⁸, R¹⁰ are each independently selected from the group consisting of:
—H, and —(C₁-C₁₂)-alkyl,
wherein the alkyl groups mentioned are optionally substituted, and
wherein at least R⁶ or R¹⁰ is —H.

8. The process according to claim 1, wherein the first phenol is the same as the second phenol.

9. The process according to claim 1, wherein the selenium dioxide is added in process step c) in a molar ratio of from 0.25 to 1.5 based on a total sum of the first and second phenols.

10. The process according to claim 1, wherein the first phenol in process step a) is a compound of the general formula III: 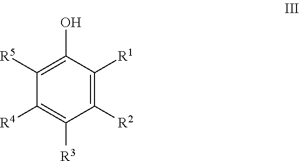
wherein R¹, R², R³, R⁴, R⁵ are each independently selected from the group consisting of:
—H, —(C₁-C₁₂)-alkyl, —O—(C₁-C₁₂)-alkyl, —(C₆-C₂₀)-aryl, —O—(C₆-C₂₀)-aryl, -halogen, and —OC═O—(C₁-C₁₂)-alkyl,
wherein two adjacent radicals are optionally joined to one another to form a fused system,
wherein the alkyl and aryl groups mentioned are optionally substituted, and
wherein R³ is —H.

11. The process according to claim 10, wherein R¹, R², R³, R⁴, R⁵ are each independently selected from the group consisting of:
—H, —(C₁-C₁₂)-alkyl, —O—(C₁-C₁₂)-alkyl, —(C₆-C₂₀)-aryl, and —O—(C₆-C₂₀)-aryl,
wherein the alkyl and aryl groups mentioned are optionally substituted, and
wherein R³ is —H.

12. The process according to claim 10, wherein R¹, R³, R⁵ are each independently selected from the group consisting of:
—H, and —(C₁-C₁₂)-alkyl,
wherein the alkyl groups mentioned are optionally substituted, and
wherein R³ is —H.

13. The process according to claim 10, wherein the second phenol in process step b) is a compound of the general formula IV: 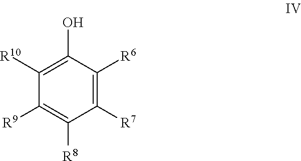
wherein R⁶, R⁷, R⁸, R⁹, R¹⁰ are each independently selected from the group consisting of:
—H, —(C₁-C₁₂)-alkyl, —O—(C₁-C₁₂)-alkyl, —(C₆-C₂₀)-aryl, —O—(C₆-C₂₀)-aryl, -halogen, and —OC═O—(C₁-C₁₂)-alkyl,
wherein two adjacent radicals are optionally joined to one another to form a fused system,
wherein the alkyl and aryl groups mentioned are optionally substituted, and
wherein R⁸ is —H.

14. The process according to claim 13, wherein R⁶, R⁷, R⁸, R⁹, R¹⁰ are each independently selected from the group consisting of:
—H, —(C₁-C₁₂)-alkyl, —O—(C₁-C₁₂)-alkyl, —(C₆-C₂₀)-aryl, and —O—(C₆-C₂₀)-aryl,
wherein the alkyl and aryl groups mentioned are optionally substituted, and
wherein R⁸ is —H.

15. The process according to claim 13, wherein R⁶, R⁸, R¹⁰ are each independently selected from the group consisting of:
—H, and —(C₁-C₁₂)-alkyl,
wherein the alkyl groups mentioned are optionally substituted, and
wherein R⁸ is —H.

16. The process according to claim 13, wherein the first phenol is the same as the second phenol.

17. The process according to claim 13, wherein the selenium dioxide in process step c) is added in a molar ratio of from 0.25 to 1.5 based on a total sum of the first and second phenols.

18. The process according to claim 1, wherein the 2,2′-selenobiaryl ether or 4,4′-selenobiaryl ether is selected from the group consisting of
bis(3,5-dimethyl-2-hydroxyphenyl)selenium 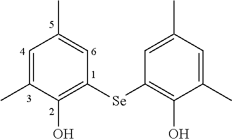
bis(3-tert-butyl-5-methyl-2-hydroxyphenyl)selenium 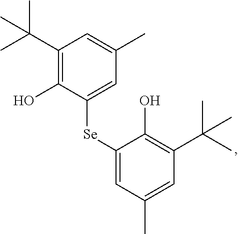 [SMILES: Cc1cc([Se]c2cc(C)cc(C(C)(C)C)c2O)c(O)c(C(C)(C)C)c1]
bis(3,5-Di-tert-butyl-2-hydroxyphenyl)selenium 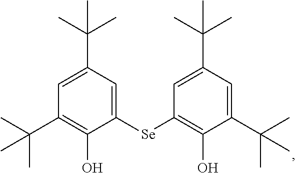 [SMILES: CC(C)(C)c1cc([Se]c2cc(C(C)(C)C)cc(C(C)(C)C)c2O)c(O)c(C(C)(C)C)c1]
bis(3-Chloro-6-hydroxy-5-isopropyl-2-methylphenyl)selenium 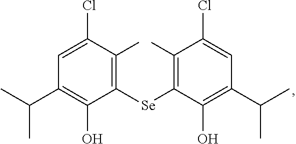 [SMILES: Cc1c(Cl)cc(C(C)C)c(O)c1[Se]c1c(C)c(Cl)cc(C(C)C)c1O]
bis(3-Chloro-6-hydroxy-5-methylphenyl)selenium 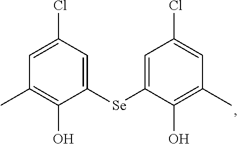
bis(2-Hydroxy-3-methoxy-5-methylphenyl) selenium 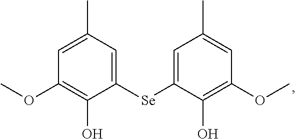
and
bis(3,5-Dimethyl-4-hydroxyphenyl)selenium

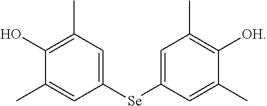 [SMILES: Cc1cc([Se]c2cc(C)c(O)c(C)c2)cc(C)c1O]

19. A compound of the formula:

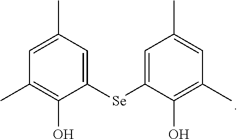

20. A compound of the formula: 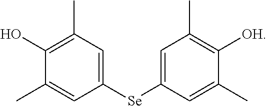 [SMILES: Cc1cc([Se]c2cc(C)c(O)c(C)c2)cc(C)c1O]
